(19) United States
(12) Patent Application Publication
(10) Pub. No.: US 2011/0130693 A1
(43) Pub. Date: Jun. 2, 2011

(54) AVE-EX

(76) Inventor: Melissa Vosche' Rutland Avery, Puryear, TN (US)

(21) Appl. No.: 12/804,566

(22) Filed: Dec. 1, 2009

(51) Int. Cl. A61F 5/37

(52) U.S. Cl. 602/20

(57) ABSTRACT

The AVE-EX is an orthotic device invented to prevent rapid abduction of the wearer's shoulder joint to preserve the soft tissue components (i.e. rotator cuff) lying underneath from tearing/re-tearing minimally, moderately or completely in order for the wearer to safely participate in high fall risk activities.

The title of the invention is, the AVE-EX, which stands for The Avery Extender. This device extends the life of the soft tissue components of the joint it overlies.

### BACKGROUND OF THE INVENTION

I came up with this invention when I injured my right rotator cuff in a snow boarding fall Feb. 14, 2009 high moderately tearing between 50 and 95% of my rotator cuff inferior fibers. In June, I went through right rotator cuff arthroscopy and during physical therapy starting asking both my surgeon and physical therapists how I could obtain an orthotic to wear while snow skiing this winter in order to prevent reinjuring what I had worked so hard at having debrided and rebuilding/strengthening. I was told by three professionals that what I was requesting and distinctly describing did not exist. Being a physician myself, thereby intimately knowing the anatomy, I decided to invent the product and make it useful for nearly all sport enthusiasts and others in the general population. I wanted to make it lightweight, wearable and able to be placed into new and existing garments. This product is specifically meant to radically diminish rapid abduction of the person's arm which is the common, precise mechanism of how I tore my rotator cuff and would still be at risk for tearing it in again without the use of my invention during future sports activities.

### BRIEF SUMMARY OF THE INVENTION

The AVE-EX came to be as a method whereby I could participate in fall risk sports after my surgeon cleared me and not be in as high a risk to re-injure my right rotator cuff or injure my left rotator cuff while snow skiing or snow boarding.

The invention is a small, lightweight, portable device that prevents rapid abduction of the wearer's left or right arm perilously putting each rotator cuff at risk of tearing moderately to fully resulting in pain, loss of work performance and costly surgery with copious commiserate physical therapy appointments.

### BRIEF DESCRIPTION OF THE SEVERAL VIEWS OF MY DRAWINGS

FIG. 1. Anterior/Posterior view in full adduction: the product is in a 90 degree angle anatomically correct position either for the right or left shoulder

FIG. 2. Anterior/Posterior view in maximum allowed abduction: (note the hydraulic system has been utilized to illicit controlled abduction to 120 degrees)

FIG. 3. Lateral view: adducted

FIG. 4. Lateral view: abducted to 120 degrees

FIG. 5. “Birdseye view” or Superior view, left shoulder: abduction

FIG. 6. “Birdseye view” or Superior view, left shoulder: adduction to 120 degrees

FIG. 7. “Birdseye view” or Superior view, right shoulder: abduction

FIG. 8. “Birdseye view” or Superior view, right shoulder: adduction to 120 degrees

### DETAILED DESCRIPTION OF THE INVENTION

This device is created for people who want to prevent new injury, postoperative recurrent injury or worsening of a prior minimal injury to the rotator cuff on either the left or the right side of the wearer during physical activity whereby they may be at risk for falling.

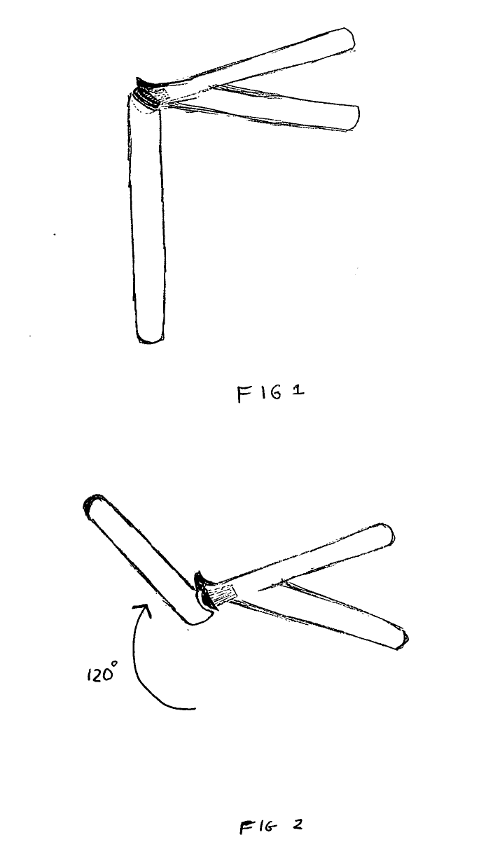

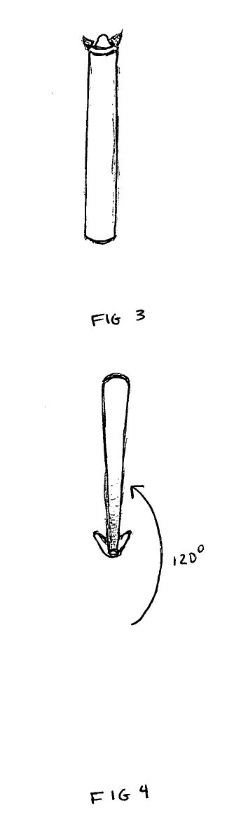

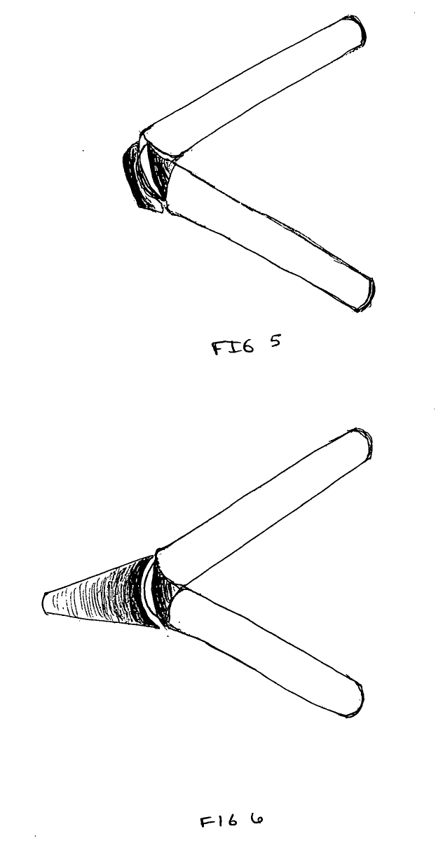

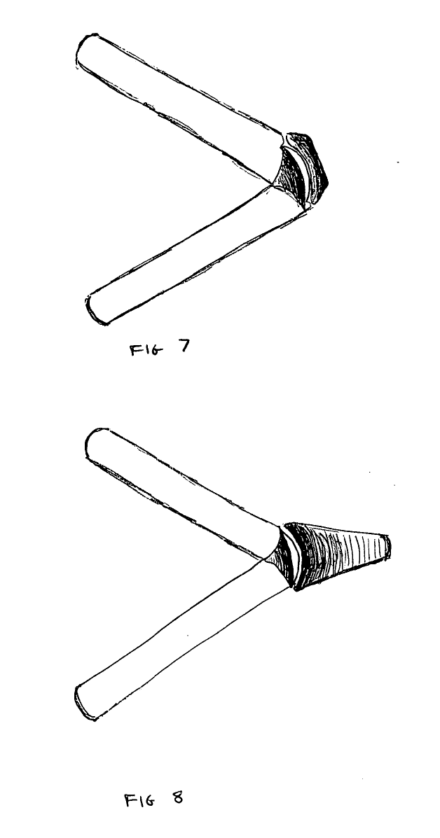

Injury prevention dictates that the device will allow necessary range of motion from 0 to 120 degrees in abduction (see FIGS. 1-8.) This is a unique invention as the product is meant to be placed into new or existing apparel versus the traditional bulky, strapped-on apparatus. The invention can be utilized by being located in a sewn in pocket of material in new clothing and via Velcro® to pre-owned clothing. The product will be made of lightweight titanium. The hinge above the person's joint will have a small hydraulic system in order to slow down momentum from a fall to prevent tear of the inferior portion of the rotator cuff fibers. I propose to have a biomechanical engineer or orthotic company to produce the device. The device will be made to anatomic dimensions to fit a pediatric or adult left or right shoulder. The device initially was created for snow skiers and is also applicable to nearly all sports enthusiasts and additionally for the geriatric elderly fall risk population.

No other invention to date targets this common mechanism of injury and is as functional and wearable to continue performing high risk sport/life activities. Further, no prior invention discards use of complex, cumbersome layering of fabrics, elastics, metals, motors, etc. Lastly, no other product is designed to work with new or existing apparel such as this interchangeable device.

1. Specifically, the AVE-EX will help to extend the life of the soft tissue fibers of the wearers' left or right sided rotator cuff to prevent new injury, postoperative recurrent injury or worsening of a prior minimal injury during physical activities including sports where falling is a distinct likelihood. This device will help to prevent injury as described by transforming rapid uncontrolled abduction into controlled abduction up to 120 degrees via a lightweight wearable combined titanium and hydraulic system. The device will be able to be placed into new garments via a sewn in pocket or into existing apparel via Velcro®.
